(19) United States
(12) Patent Application Publication
(10) Pub. No.: US 2026/0256413 A1
(43) Pub. Date: Sep. 3, 2026

(54) MULTIMODAL SEIZURE SENSING

(71) Applicant: PURDUE RESEARCH FOUNDATION, West Lafayette, IN (US)

(72) Inventors: Pedro P. Irazoqui, Baltimore, MD (US); Jay Vatsal Shah, West Lafayette, IN (US); Trevor Donald Meyer, Baltimore, MD (US); Vivek Ganesh, Baltimore, MD (US); Swagat Bhattacharyya, Morgantown, WV (US); Yi-Fang Hsiung, West Lafayette, IN (US)

(21) Appl. No.: 18/857,956

(22) PCT Filed: Apr. 26, 2023
(86) PCT No.: PCT/US2023/020047

§ 371 Date: Oct. 18, 2024

Related U.S. Application Data
(60) Provisional application No. 63/334,832, filed on Apr. 26, 2022.

(51) Int. Cl. A61B 5/00; A61B 5/11; A61B 5/296; A61B 5/313; A61B 5/389

(52) U.S. Cl. CPC A61B 5/4094; A61B 5/1118; A61B 5/1123; A61B 5/296; A61B 5/313; A61B 5/389; A61B 5/6823; A61B 5/6824; A61B 5/6828

(57) ABSTRACT

This disclosure describes systems, apparatuses, and techniques that use of multiple sensing modalities (multimodal sensing) in parallel to improve the detection, classification, and evaluation of seizures in subjects (e.g., mammalian subjects, such as humans). In some implementations, a wearable device includes a flex sensor configured, an inertial motion unit (IMU), and a plurality of surface electromyogram (sEMG) electrodes configured to be placed approximate to a muscle of a subject. The device also includes a microprocessor. The operations performed by the microprocessor include determining multimodal movement data of the muscle, where the multimodal movement data specifies at least an acceleration associated with movement of the muscle, an angular velocity associated with movement of the muscle, and sEMG activity associated with the movement of the muscle. The operation further includes determining whether the multimodal movement data indicates a possible seizure, and provide data based on the determination for output.

### CROSS-REFERENCE TO RELATED APPLICATIONS

This application claims the benefit of U.S. Provisional Application No. 63/334,832, filed Apr. 26, 2022, the disclosure of which is incorporated herein by reference in its entirety.

### TECHNICAL FIELD

This disclosure generally relates to seizure detection and, in particular, to wearable devices for seizure detection.

### BACKGROUND

Epilepsy, a brain disorder resulting in seizures, affects more than 65 million people worldwide. There are an estimated 3.5 million Americans-including 470,000 children-living with epilepsy. In approximately 35% of cases, seizures cannot be controlled by medication, and uncontrolled seizures are the primary risk factor for sudden, unexpected death from epilepsy (SUDEP). SUDEP is a sudden, unexpected, non-traumatic death, occurring in benign circumstances in an individual with epilepsy, with or without evidence that a seizure has occurred. In the US, the annual mortality rate for SUDEP in patients with epilepsy is approximately 1.2/1000 translating to ~4000 unexpected deaths a year as a low estimate, within an at-risk population well in excess of 1 million. Due to SUDEP, 101,000 years of potential life are lost annually, second only to stroke. While all epileptic seizures pose a risk, unmonitored generalized tonic-clonic seizures (GTCS) are of particular concern. GTCS are the most common afebrile seizure type in the general population and also the most dramatic of all seizures. Monitoring seizures and other associated dysfunctions is important to assessing the severity of epilepsy and mitigate subsequent risk of SUDEP.

### SUMMARY

This disclosure describes systems, apparatuses, and techniques that use of multiple sensing modalities (multimodal sensing) in parallel to improve the detection, classification, and evaluation of seizures in subjects (e.g., mammalian subjects, such as humans). As discussed herein, “multimodal” sensing refers to the use of multiple types of sensors or sensing modalities to capture different aspects of a given physiological phenomenon associated with a seizure event. For example, in treating epileptic conditions, “multimodal” sensing may involve combining data from embedded sensors of a single wearable device, data from multiple discrete wearable devices, or a combination thereof. Multimodal sensing may also involve combing sensed data with data from imaging or other diagnostic tests, to gain a more comprehensive understanding of a subject's health status.

Multimodal sensing permits seizure detection and classification through the collection and correlation of multiple physiological signals that permit enhanced characterization of muscle movements that may result from seizure-related conditions. This improves seizure detection by enabling more accurate detection (e.g., reducing the number of false-positive and/or false-negative detections) and broader seizure classification (e.g., distinguishing between different types of detected seizures). For example, multimodal sensing can be utilized to monitor seizure-induced cardiac or respiratory dysfunctions known to occur prior to or after a seizure event.

The multimodal sensing techniques disclosed herein may be used to monitor various types of seizures and/or used to treat various types of epileptic disorders. Examples of seizures that may be detected using multimodal sensing include generalized tonic-clonic seizures, absence seizures, myoclonic seizures, atonic seizures, and partial seizures, among others. Multimodal sensing can also be used to monitor seizures that occur amongst various types of epileptic disorders, such as idiopathic generalized epilepsy, temporal lobe epilepsy, frontal lobe epilepsy, juvenile myoclonic epilepsy, Lennox-Gastaut syndrome. Multimodal sensing may be used to predict SUDEP, a complication of epilepsy, where a person with epilepsy dies suddenly and unexpectedly, usually during or immediately after a seizure, and with no obvious cause of death identified during autopsy.

As described herein, multimodal sensing can be accomplished through use of a wearable with embedded sensors configured to monitor different physiological conditions associated with seizure events, such as cardiorespiratory dysfunction and associated muscle movement data. For example, the monitored data may indicate cardiac and respiratory signals that are analyzed in parallel with movement data of a muscle to detect and classify seizures in outpatient environments. In this example, the movement data may specify at least an acceleration associated with muscle movement, an angular velocity associated with muscle movement, and surface electromyogram (sEMG) activity associated with muscle movement.

The wearable device may be placed on a suitable location of the subject. For example, in human subjects, the wearable device may be placed on an arm, a leg, torso, neck, among others that permit detection of muscle movement. In some implementations, the wearable device is placed on a subject to increase comfort and/or improve sensing efficiency. In some implementations, the wearable device is configured to be worn only at night while a subject is asleep. In other implementations, the wearable device is used in conjunction with a suitable attachment mechanism to permit the device being worn for longer time frames (e.g., to provide continuous seizure detection capabilities).

The wearable device may have various form factors to support the specific type of seizure detection capabilities contemplated within this disclosure. In some implementations, the wearable device is formed as a patch with an internal sensor suite. In other implementations, the wearable device is formed as a band, cuff, or a watch.

The wearable devices configured to use the multimodal sensing disclosed herein may provide several life-saving capabilities for various epileptic disorders and other disorders that may cause seizures. For example, because some disorders can result in cardiorespiratory collapse in conjunction with a seizure, reducing morbidity associated with these disorders necessitates monitoring cardiorespiratory in addition to seizure detection capabilities. A wearable device that utilizes multimodal sensing to monitor cardiorespiratory function in parallel with muscle movement can thereby more effectively detect risks of death associated with seizure events. For example, a wearable device may be configured such that, upon detecting a seizure, cardiac and respiratory dysfunctions may also be monitored to predict the risk of SUDEP. In this example, collected data may be transmitted to external devices, such as the devices of a caregiver or a healthcare provider.

In one general aspect, this disclosure includes a system. The system includes a wearable device with a flex sensor, a plurality of surface electromyogram (sEMG) electrodes, and circuitry. The circuitry is configured to measure, sEMG activity of a muscle proximate to the sEMG electrodes based on sEMG signals received from the sEMG electrodes, measure movement of the muscle based on signals received from the flex sensor, measure acceleration and angular velocity based on signals generated by an inertial motion unit (IMU), and wirelessly communicate the measured sEMG activity, the measured muscle movement, and the measured acceleration and angular velocity.

One or more implementations may include the following optional features. For example, in some implementations, the system further includes a remote device configured to receive the inertial motion measurements, the measured sEMG activity, and the measured acceleration and angular velocity, determine, based on the received measurements, a seizure is occurring in a person wearing the wearable device, and output a message in response to determination of the seizure.

In some implementations, determining a seizure is occurring in a person wearing the wearable device, the processor of the remote device is further configured to identify features based on the inertial motion measurements, the measured sEMG activity, and the measured muscle movement, and classify the features as a seizure event.

In some implementations, the IMU includes a 3-axis gyroscope and a 3-axis accelerometer.

In some implementations, in measuring sEMG activity of a muscle proximate to the sEMG electrodes, the circuitry is further configured to receive the signals from the sEMG electrodes and filter the signals, wherein the filtered signals from the sEMG electrodes have a frequency between 100 Hz and 300 Hz.

In some implementations, the circuitry is further configured to determine the root-mean-square (RMS) of the filtered signals.

In some implementations, the circuitry is further configured to generate an electrical signals based on the voltage of the flex sensor and filter the signals, wherein the filtered signals from the flex sensor signals have a frequency between 0.1-50 Hz.

In some implementations, the circuitry is further configured to generate a first acceleration vector parallel to the plane of the bed and a second acceleration vector perpendicular to the plane of the bed.

In some implementations, the flex sensor is positioned in between the two electrodes, wherein the flex sensor and electrodes are flexible and conform to a surface of an arm along a muscle.

In some implementations, the flex sensor is elongated at curves about the bicep along a direction substantially perpendicular to a long head of the muscle and the sEMG electrodes are oriented in a direction parallel to the long head of the muscle.

In some implementations, the wearable device further includes a shuttle which receives the sEMG electrodes, flex sensor, and circuitry.

In some implementations, the wearable device further includes a housing configured to conform and flex to a muscle, wherein the shuttle is disposed in the housing and at least a portion of the sEMG electrodes outside of the housing for contact with the arm.

In some implementations, the flex sensor is disposed inside of the housing, wherein flexure of the housing causes flexure of the flex sensor.

In some implementations, the device includes an adhesive layer attached to an outer surface of the housing, wherein the adhesive layer is configured to attach the wearable device to an arm.

In some implementations, the muscle is a bicep of an arm.

In another general aspect, a wearable device includes a flex sensor configured to collect one or more first signals. The wearable device also includes an inertial motion unit (IMU) configured to collect one or more second signals, a plurality of surface electromyogram (sEMG) electrodes configured to (i) be placed approximate to a muscle of a subject and (ii) collect one or more third signals, and a microprocessor configured to perform operations. The operations include determining multimodal movement data of the muscle based on the one or more first signals, the one or more second signals, and the one or more third signals, wherein the multimodal movement data specifies at least an acceleration associated with movement of the muscle, an angular velocity associated with movement of the muscle, and sEMG activity associated with the movement of the muscle, determining whether the multimodal movement data indicates a possible seizure, and providing, for output, data indicating whether the movement indicates a possible seizure.

One or more implementations may include the following optional features. For example, in some implementations, the muscle includes an arm muscle.

In some implementations, the muscle includes a leg muscle.

In some implementations, the muscle includes a torso muscle.

In some implementations, determining whether the multimodal movement data indicates a possible seizure includes processing the first set of signals, the second set of signals, and the third set of signals, identifying a set of features associated with the multimodal movement data based on processing the first set of signals, the second set of signals, and the third set of signals, determining that the set of features include a predetermined feature associated with a seizure condition, and determining that the multimodal movement data indicates the possible seizure based on determining that the set of features include the predetermined feature.

In another general aspect, a method includes placing the wearable device on a subject, wherein the wearable device comprises (i) a flex sensor, (ii) an inertial motion unit (IMU) sensor, and (iii), a plurality of surface electromyogram (sEMG) electrodes. The method also includes receiving data indicating (i) one or more first signals from the flex sensor, (ii) one or more second signals from the IMU sensor, and (iii) one or more third signals from the plurality of sEMG electrodes, determining multimodal movement data of the muscle based on the one or more first signals, the one or more second signals, and the one or more third signals, wherein the multimodal movement data specifies at least an acceleration associated with movement of the muscle, an angular velocity associated with movement of the muscle, and sEMG activity associated with the movement of the muscle, determining that the multimodal movement data indicates a possible seizure, and providing, for output, data indicating the possible seizure.

One or more implementations may include the following optional features. For example, in some implementations, placing the wearable device on the subject includes placing the wearable device on an arm of the subject.

In some implementations, placing the wearable device on the subject comprises placing the wearable device on a leg of the subject.

In some implementations, placing the wearable device on the subject comprises placing the wearable device on a torso of the subject.

### BRIEF DESCRIPTION OF DRAWINGS

FIG. 1 illustrates a first example of a system.

FIG. 2 illustrates a first example of a wearable device.

FIG. 3 illustrates an example of sEMG signal conditioning circuitry.

FIG. 4 illustrates an example of flex sensing circuitry and logic.

FIG. 5 illustrates an example of a flowchart for logic to discriminate between motion parallel and perpendicular to the plane of the bed using a 3 axis accelerometer and a 3-axis gyroscope.

FIG. 6 illustrates a flowchart of an example of seizure detection logic based on multimodal input.

FIGS. 7A-B illustrate a second example of the wearable device.

FIG. 8 illustrates an example of a shuttle for a wearable device.

FIG. 9 illustrates a top view of a wearable device with annotations for flexible portions.

FIG. 10 illustrates a perspective view of a wearable device.

FIG. 11 illustrates an example of a wearable device positioned on an arm.

FIG. 12 illustrates an exploded view of an example of a wearable device.

FIG. 13 illustrates a second example of a system.

FIGS. 14A-14B illustrate results of a usability study conducted using examples of the wearable device configured to provide a multimodal seizure sensor array.

FIG. 15 illustrates results of a case study evaluating seizure detection capabilities of a wearable device configured to provide a multimodal seizure sensor array.

FIGS. 16A-C illustrate configurations of an adhesive pad for placing a wearable device onto a body of a subject.

FIG. 17 illustrates an example of a process for detecting a possible seizure using a wearable device configured to provide a multimodal seizure sensor array.

The embodiments may be better understood with reference to the following drawings and description. The components in the figures are not necessarily to scale. Moreover, in the figures, like-referenced numerals designate corresponding parts throughout the different views.

### DETAILED DESCRIPTION

Current standard of care for epilepsy patients is to visit an epilepsy monitoring unit (EMU) to determine ways to control their seizures, either via an implantable device or through surgical resection. However, only 2-4% of the total US candidates undergo surgery annually, with wait times averaging 1-5 months just to enter an EMU. Some of this long wait time can be attributed to psychogenic non-epileptic spells (PNES). Between 20-40% of EMU admissions result in a diagnosis of PNES, a condition that manifests seizure-like activity but is not the same as epileptic seizures [0]. Screening for these potential patients ahead of time and shortening their stay in the EMU can improve the long wait times associated with EMUs. Access to timely treatment to control seizures is further delayed by the time taken to refer these patients to epilepsy specialists prior to EMU admission. Once admitted, seizures are often detected and recorded by using multiple signal processing means, e.g., video-EEG, CT- and MRI-scanning, motion detection, etc. This process is not only very time consuming, but also requires a lot of data analysis to determine the characteristics of the seizure. Nevertheless, epilepsy patients are at high risk of SUDEP not only as they wait to receive treatment but also for as long as their seizures remain uncontrolled. Hence, there is a need for remote monitoring of epilepsy patients during sleep when caretakers are usually not in the vicinity. Anecdotal evidence provided by patients regarding seizure severity and frequency is often insufficient for clinicians to assess treatment strategies, so quantitative measurements in the form of a non-invasive wearable system are distinctly advantageous. Although electroencephalogram (EEG) is the gold-standard for seizure detection in the clinical setting, EEGs are not well-suited for daily, residential use due to the difficulty of mounting the numerous EEG electrodes with the necessary accuracy. Current non-EEG seizure detection methods often measure acceleration, rotation rate, electrodermal activity, or myoelectric activity of limbs/appendages. These methods are usually embodied as a cuff, patch, or watch. Some seizure detectors use mattress-based pressure sensors to detect seizing or microphones to detect ictal cries. Multimodal sensing schemes, which use a combination of two or more sensors, have demonstrated improved specificity (lower false detection rate) over unimodal approaches. Most current non-EEG seizure detectors are sensitive only to GTCS; while monitoring GTCS is important, it is only one of 7 different types of epileptic seizures observed in humans and as such, there is demand for a more generalized seizure detection system in addition to detecting and differentiating PNES from seizures.

Accordingly, there are disclosed a system and methods for detecting seizure activity. The system may include a wearable device having a multi modal seizure sensor array. The multimodal seizure sensor array may include an accelerometer, a gyroscope, a surface electromyogram (sEMG), a flex sensor, and related circuitry. This sensor array will be used to detect seizures, including but not limited to generalized tonic-clonic seizures, in epilepsy patients. This technology uses three sensor systems: an inertial measurement unit (IMU) containing an accelerometer and gyroscope, surface electromyogram, and flex sensing synergistically to estimate the myoelectric (muscle) potentials and movements during a motor seizure. Our sensing technology promises better sensitivity and specificity over existing solutions, potentially improving seizure detection accuracy.

An example of a technical advancement achieved by the systems and methods described below is the combination of modalities selected for seizure detection. Flex sensing in combination with sEMG, accelerometry and gyroscopy promise to deliver low false positives and improved accuracy for seizure detection, in a wearable, low power implementation.

Another example of a technical advancement achieved by the systems and methods described below may be that of improved specificity by localizing motor movement; the flex sensor and sEMG estimate local muscle movements while the IMU measures global/generalized body movements.

A third example of a technical advancement achieved by the systems and methods described involve improving sensitivity and specificity by estimating the local muscle movement via two sensors: the flex sensor and the sEMG. sEMG has been used in other non-EEG seizure detectors, and the dominant energy content in sEMG signals lies between 30 Hz and 200 Hz. PNES has a spectral peak in the sub-30Hz range, however sEMG readings in this band can be contaminated by motion artifacts. Additionally, sEMG readings within the 50 Hz-60 Hz band can be contaminated by coupled powerline noise. To mitigate this, we use measurements from a flex sensor attached to the muscle to estimate the low-frequency sEMG content; flex-sensors encounter less noise contamination than sEMG at low frequencies. Hence, we reserve our sEMG for the analysis of high-frequency biopotential signals in the 100 Hz-300 Hz range, using aggressive filtering to attenuate components outside the 100 Hz-300 Hz range (resulting in a higher SNR than a typical sEMG system). The larger bandwidth provided by this system can enable better seizure detection and potential differentiation of PNES by utilizing the ratio between the RMS power of the high-frequency and low-frequency myoelectric signal estimates to differentiate tonic seizures, generalized tonic-clonic seizures (GTCS), and PNES.

A fourth example of a technical advancement achieved by the systems and methods described is a novel switching RMS envelope detector topology to achieve higher accuracy.

The system and methods described herein offer improvements over existing market solutions. Additional benefits, efficiencies, and improvements over existing market solutions are made evident in the system and methods described below.

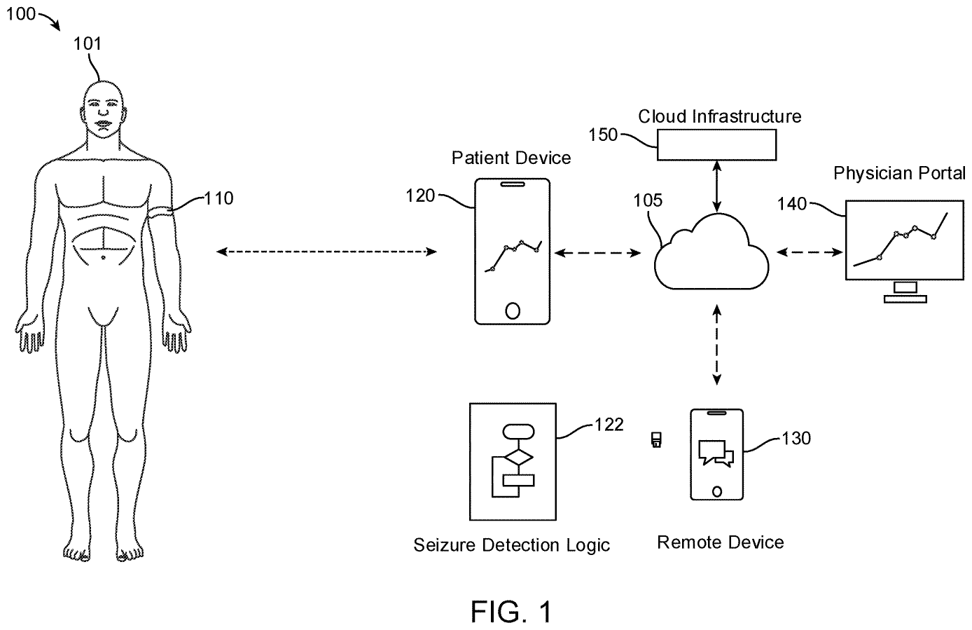

FIG. 1 illustrates a first example of a system. The system 100 may include a wearable device. The wearable device 110 may be affixed to a person's body 101 and collect multiple physiological signals including, for example, sEMG, flex sensing, accelerometry, angular velocity, ECG, respiration, oxygen saturation, and others. The wearable device 110 may communicate the collected data to external endpoint(s), such as a patient device 120. The wearable device 110 may condition the signals obtained by sensor(s) (e.g., integrated sensors, wearable sensors) and obtain physiological measurements communicated to the patient device 120. The measurements may be fixed or time varying. In some examples, the physiological measurements may be streamed in real time.

The patient device 120 may include a device capable of wirelessly communicating with the wearable device. In some examples, the patient device 120 may include a phone or mobile device with a display, as illustrated in FIG. 1. Alternatively or in addition, the patient device 120 may include a device in a fixed or semi-fixed location.

Seizure detection logic 122 may evaluate information communicated by the wearable device 110 to determine or predict whether a patient 101 wearing the wearable device 110 is having a seizure. In some examples, the patient device 120 may include the seizure detection logic 122, though it is possible for the seizure detection logic 122 to be included on other devices which receive the physiological measurements generated by the wearable device 110.

The system 100 may further include cloud infrastructure 150. The cloud infrastructure 150 may include one or more servers (either physical or virtual) which receive information from the patient device 120. For example, data collected by the patent device 120 may be communicated to the cloud infrastructure 150. In some examples, the system 100 may further include one or more physical portals where physicians will have access to recorded data through a physician portal. In some examples, the patient device 120 and/or the cloud infrastructure 150 may include logic which alerts a caretaker when a seizure is detected.

It should be appreciated that other embodiments are possible. For example, the wearable device 110 may communicate alerts, sensor data and/or seizure collection data directly with the patient device 120, the cloud infrastructure 150, the physical portal 140, or a combination thereof. Alternatively or in addition, the cloud infrastructure 150 may include the seizure detection and/or the alert logic.

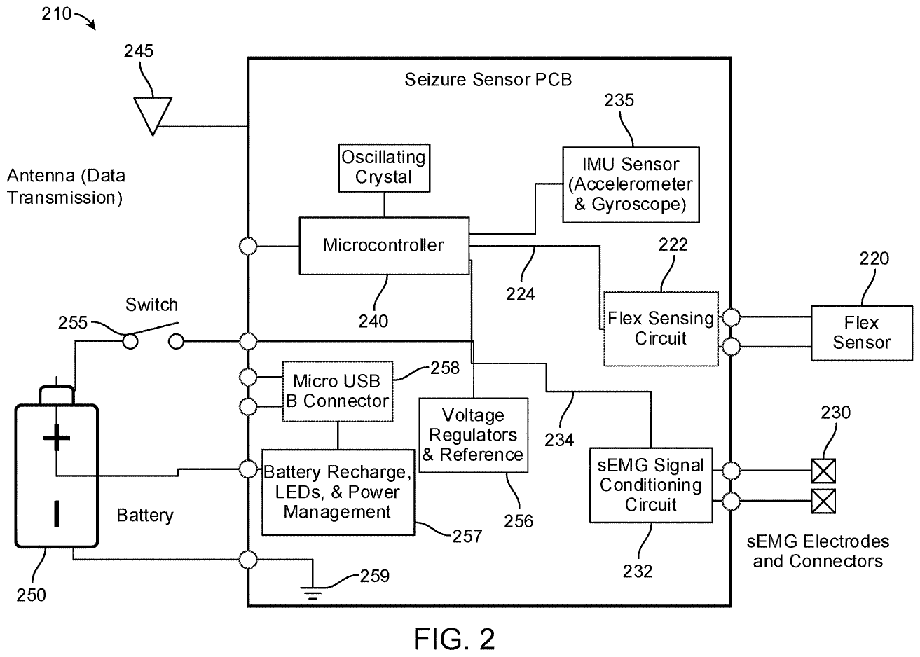

FIG. 2 illustrates an example of a wearable device 210. In various examples, the wearable device 210 may include a flex sensor 220 and surface electromyography (sEMG) electrode(s) 230.

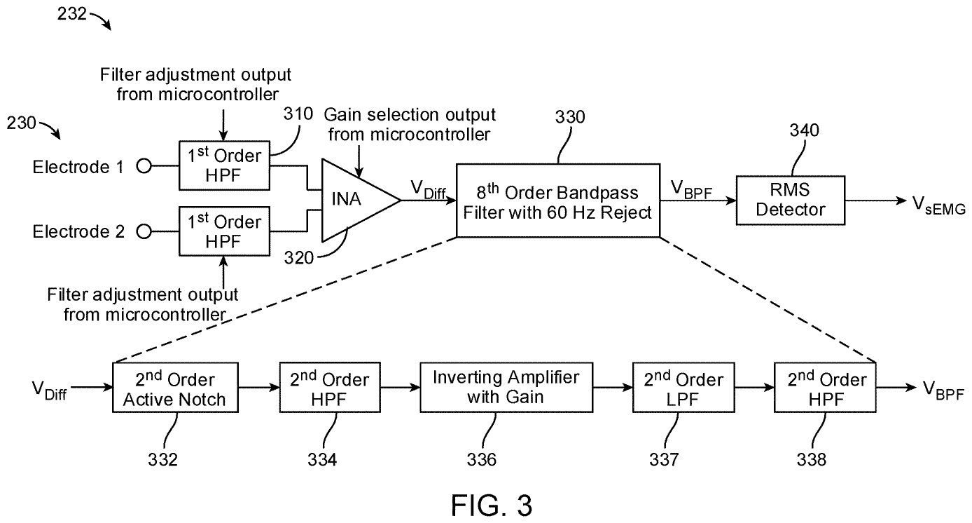

The sEMG electrodes 230 may generate physiological signals representative of the electrical activity (voltage) of the underlying muscle group(s) at rest and during activity. The sEMG electrodes 230 may provide signals which are conditioned with sEMG signal conditioning circuitry 232. The sEMG circuitry is discussed further described below and exemplified in FIG. 3.

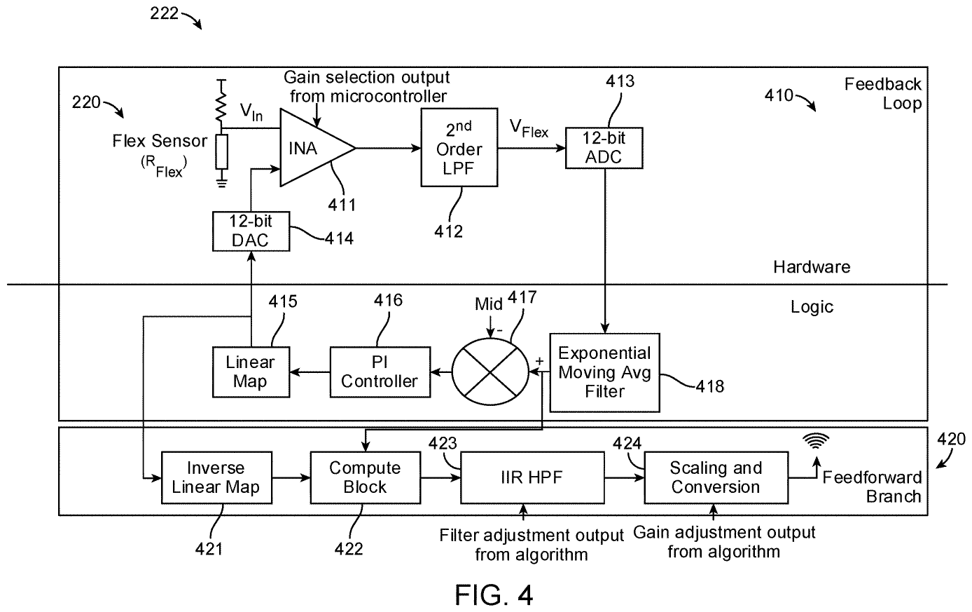

The flex sensor 220 may generate physiologic signals representative of the strain of the underlying muscle group(s) via a resistance measurement. The signals generated by the flex sensor 220 may be conditioned with flex sensing circuitry 222, which is discussed further below and exemplified in FIG. 4.

The wearable device 210 may include an inertial motion unit (IMU) 235. The IMU 235 may include, for example, an accelerometer and/or a gyroscope. Accelerometry combined with gyroscopy is a sensitive means of seizure detection. Nevertheless, some historical work leveraging magnetometers in addition to accelerometry and gyroscopy have demonstrated a higher specificity. Indeed, movement in the horizontal plane of the bed is characteristic of a tonic seizure, and use of a magnetometer allows for a better estimation of the horizontal motion. However, the system and methods described herein discriminate between motion parallel and perpendicular to the plane of the bed using only a 3-axis accelerometer and a 3-axis gyroscope, eliminating the need for a magnetometer and reducing the power draw of the wearable. In various embodiments, the system and methods describe herein a complementary filter to estimate the instantaneous direction of the gravity vector and use vector projections to find the acceleration parallel and perpendicular to the plane of the bed. This allows for effective seizure detection in a low-power budget.

Accordingly, a technical advancement provided by the system and method described herein is the ability to discard the use of a magnetometer and rely on using just 3-axis accelerometry and 3-axis gyroscopy to achieve equal/better accuracy on a lower power budget. For example, the logic described herein for determining the gravity vector eliminates the need for a magnetometer while also providing better performance (power) and accuracy. As described herein, the IMU 235 may estimate orientation (which is the “pitch” and “roll” of the sensor), and then uses the orientation information to estimate the gravity unit/direction vector. This unit vector is then used for the projections.

The wearable device 210 may further include a microcontroller 240. The microcontroller may receive conditioned sEMG signals 234, conditioned flex sensor signals 224, and signals from the IMU 235. An oscillating crystal is used to provide stable clock signals for digital electronics in the wearable device 210.

The microcontroller 240 may package these signals and wirelessly transmit the data. For example, the microcontroller 240 may cause the data to be communicated to the patient device 120 (shown in FIG. 1) using antenna 245. Alternatively or in addition, the microcontroller 240 may communicate the data over a network to the cloud infrastructure or some other remote endpoint.

The wearable device 210 may further include a battery 250 and switch 255 for power, an antenna 245 for wireless data transmission. In some examples, the powering circuitry may include various integrated circuits (ICs) for battery recharging and voltage generation, for example, one or more voltage regulators or reference voltages 256. LEDs and switches 257, connectors 258, and grounds 259.

FIG. 3 illustrates an example of the sEMG signal conditioning circuitry 232. By way of example, each signal from the sEMG electrodes 230 may pass through a 1^{st} order high pass filter 310 and then feeds into an instrumentation amplifier (INA) 320. The output of the INA 320 may be filtered through an 8^{th} order band pass filter 330 with 60 Hz noise rejection and is then passed into a novel RMS envelope detection circuit 340. The 8^{th} order band pass filter 330 may condition the signal to allow frequencies in the 100-300 Hz range and may include a 2^{nd} order 60 Hz notch filter 332, two 2^{nd} order high pass filters 334 and 338, a 2^{nd} order low pass filter 337, and an inverting amplifier 336. Utilizing a 60 Hz notch filter 332 within the bandpass filter 330 facilitates sufficient attenuation of coupled powerline noise while not necessitating a large increase in the bandpass filter order.

The instantaneous power of an sEMG signal can indicate the degree of muscle activation. The RMS envelope of a signal is an estimate of the signal's instantaneous power; hence, sEMG measurements during GTC seizures are known to have high RMS values. Note that “RMS envelope detection” should be differentiated from “envelope detection” (oft-used in demodulation schemes), which does not provide a good power estimate due its sensitivity to extrema.

The system and methods described herein provide an improved RMS envelope detector comprising a peak detector based on the mathematical principles and a custom ripple filter that would be appreciated by a person of ordinary skill in the art. For example, an RMS detector fabricated on a custom application-specific integrated circuit (IC) consisting of a peak detector and a nonlinear ripple filter may be used as well as a mathematical framework for analyzing and tuning the RMS detector performance. RMS detectors typically have complex design tradeoffs among temporal tracking accuracy, output ripple content, and input carrier frequency. A key advantage of some designs is that the user could intelligently adjust these design tradeoffs for their intended application. Circuitry has been developed based on this theory of operation, implemented and tuned for seizure detection. The proposed circuitry may consist of a peak detector and a ripple filter. For the peak detector, the behavior of a Gm-C integrator pair with asymmetric time constants, was implemented with a passive RC integrator and a switching circuit to alternate between two different resistances. Additionally, a third-order, linear low pass filter was used for ripple filtering instead of a nonlinear first-order ripple filter. In an experimental test, our RMS detector demonstrated 53.9 dB of dynamic linear range at a carrier frequency of 200 Hz and had a modulation waveform cutoff frequency of 23 Hz (given a 200 Hz carrier waveform).

FIG. 4 illustrates an example of the flex sensing circuitry 222 and logic for interfacing with the flex sensing circuitry 222.

The flex sensor 220 may act like a variable resistor (R_{flex}) that changes in resistance as the flex sensor 220 is manipulated. Accordingly, the flex sensor 220 resistance may be interchangeably referred to as R_{flex}. The system parses the flex sensor 220 resistive divider output using a feedback loop 410 and a feedforward branch 420.

The feedback loop 410 functions independently from the feedforward branch 420 (the bottom row software system blocks) but not vice-versa. The objective of the feedback loop 410 is to condition the voltage range and the frequency content of Vᵢₙ to obtain the optimum measurement from the 12-bit ADC. The feedback loop 410 accomplishes this by 1) Centering V_{Flex} at the midpoint of the voltage supply (Mid) when Vᵢₙ is static (i.e., there is no movement causing a change in the flex sensor resistance) to lower the likelihood that a large, spontaneous jerk of the flex sensor in either direction causes V_{flex} to saturate (approach either the positive or negative supply voltage) thereby resulting in data loss, 2) greatly amplifying changes in V_{Flex} for fine measurement, and using anti-aliasing and post-quantization filters. In some implementations, the feedback loop 410 includes hardware including an INA 411, 2nd order low pass filter 412, 12-bit ADC 413, and 12-bit DAC 414. In some implementations, the feedback loop also contains necessary logic including a linear map 415, a proportional integral (PI) controller 416, a multiplexer 417, and an exponential moving average filter 418. Other implementations of the feedback loop may choose to use different hardware or logic.

The objective of the feedforward branch 420 is two-fold: 1) combine the DAC 414 code from the last time step (analogous to a coarse measurement) and the current value of V_{Flex} (analogous to a fine measurement) to compute the estimated resistance of the flex sensor (R_{Flex}), and 2) condition the range and frequency content of R_{Flex} for high-resolution transmission. In some implementations, the feedforward branch 420 includes appropriate logic, to include an inverse linear map 421, a compute block 422, a high pass filter 423, and scaling and conversion functions 424. Other implementations of the feedforward branch may choose to use different logic, or include additional hardware.

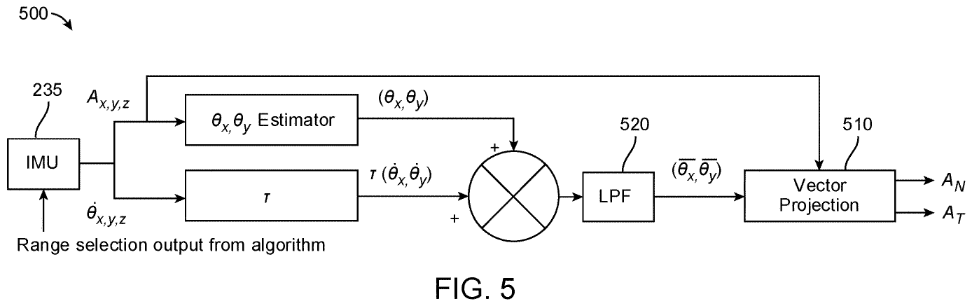

FIG. 5 illustrates an example of a flowchart 500 for logic to discriminate between motion parallel and perpendicular to the plane of the bed using a 3-axis accelerometer (Aₓ, A_{y}, A_{z}) and a 3-axis gyroscope (Θₓ, Θ_{y}, Θ_{z}) (e.g., obtained from IMU 235). Motion discrimination is performed using a vector projection algorithm 510, which accepts the acceleration vector and an estimate of the wearable angular orientation as inputs. The logic uses a complementary filter 520 to estimate the pitch (Θx) and roll (Θy) of the wearable. The complementary filter performs the following steps: (1) computation of the first two components of the instantaneous angle (θₓ, θ_{y}) of the acceleration vector from the accelerometer, (2) multiplication of the first two components of the rotational rate vector from the gyroscope by a time constant t, (3) summation of the computations from step 1 and step 2, and (3) filtering of the sum using a lowpass filter (LPF). The logic then uses vector projections 510 to find the acceleration components parallel (Aₜ) and perpendicular (Aₙ) to the reference plane of the bed. One technical advantage of this approach is that the need for a magnetometer is eliminated, thus reducing the power draw of the wearable. This allows for effective seizure detection in a low-power budget.

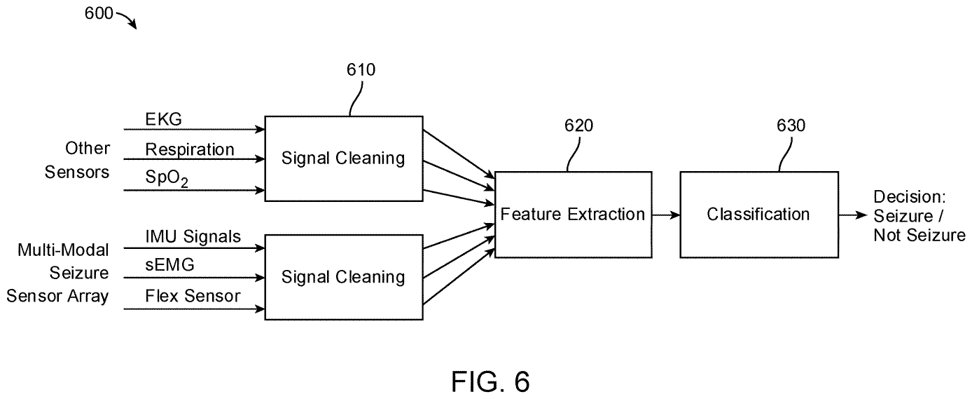

FIG. 6 illustrates a flowchart 600 for an example of seizure detection logic based on multimodal input. The seizure detection logic in FIG. 6 details the end-end processing steps in a seizure detection algorithm, including conditioning the input signal(s), analyzing its content(s) and determining an end outcome.

Data which is measured from the wearable device may be streamed in real time and processed by a seizure detection logic. First each signal will be processed to remove expected noise (e.g., “signal cleaning” 610), which includes but is not limited to high pass and low pass filtering, artifact rejection, outlier removal, interpolation, etc. This may involve using signals to verify each other, for example detecting cases of excessive motion using the IMU 235 to remove or ignore motion artifacts in other channels. Once the signals are sufficiently cleaned and acceptably free of noise, they will be passed to a feature extractor 620, which will analyze the signal to look for features that are determined to be important in detecting seizure activity. This could include but is not limited to analyzing frequency content, detecting the rate or intensity or regularity of a particular feature, correlation with an expected template, or Machine Learning-based techniques to determine the presence of important signal characteristics. Once the relevant features are extracted, they will be passed to a classifier 630, which will classify the extracted features and signal attributes as either within a normal and therefore not seizure, or abnormal/unexpected and therefore seizure.

The signal cleaning 610 or feature extraction 620 may also monitor signal quality. If the signal quality is not sufficient, for example, the signal amplitudes are too low or too high, there is too much noise present, etc., the algorithm can note these findings and signal back to the device(s) a requested change in hardware to improve signal quality. This may include but is not limited to increasing gain, changing filtering attributes, modifying resolution etc. For example, if the algorithm detects too much noise in sEMG subsystem output (e.g., by implementing a real-time spectrogram) or that amplitude is too low based on predefined thresholds, signal quality can be adjusted by using digital capacitors/potentiometer to adjust the high pass filter feeding into the input instrumentation amplifier or by adjusting the gain of the instrumentation amplifier by using a digital potentiometer. Similarly, in the flex sensing circuitry, the signal quality can be adjusted by implementing a digital potentiometer to adjust the gain of the instrumentation amplifier and by modifying the coefficients of the IIR high pass filter in firmware. Lastly, if the algorithm determines acceleration and/or angular velocity signal magnitudes are too low/high based on predefined thresholds, it can communicate with the microcontroller to update the range of the IMU.

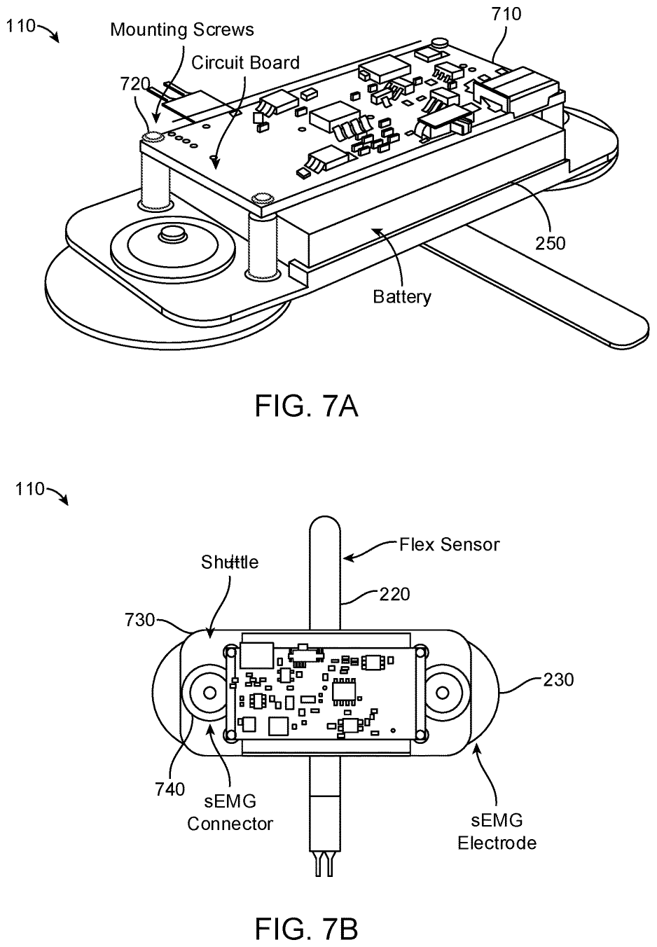

FIGS. 7A-7B illustrate a second example of the wearable device 110. FIG. 7A illustrates a perspective view and FIG. 7B illustrates a top view. The wearable device is a combination of soft and flexible materials allows a user to wear the device comfortably, as well as rigid materials, The rigid components may include a printed circuit board (PCB) 710 including circuitry, such as the circuitry described in reference to FIG. 2, a battery 250, mounting screws 720, the sEMG connectors 740 and conductive sEMG electrodes 230, and the carrier shuttle 730 used to secure the electronics and related hardware connections. The sEMG electrodes 230 can snap into the sEMG connectors 740, which can be connected to the circuit board using wires. This fully integrated device can then be cast in a soft and flexible material such as silicone, polyurethane etc. to allow the device to be wearable. The flex sensor 220 may need to be able to bend effectively for proper sensing and therefore requires a flexible housing. A soft and flexible single-use or multi-use double sided adhesive may then be used to attach the wearable device to a user's skin for monitoring. In future iterations, the rigid printed circuit board (PCB) 710 can be converted to a flexible circuit board to provide additional conforming ability to the entire device.

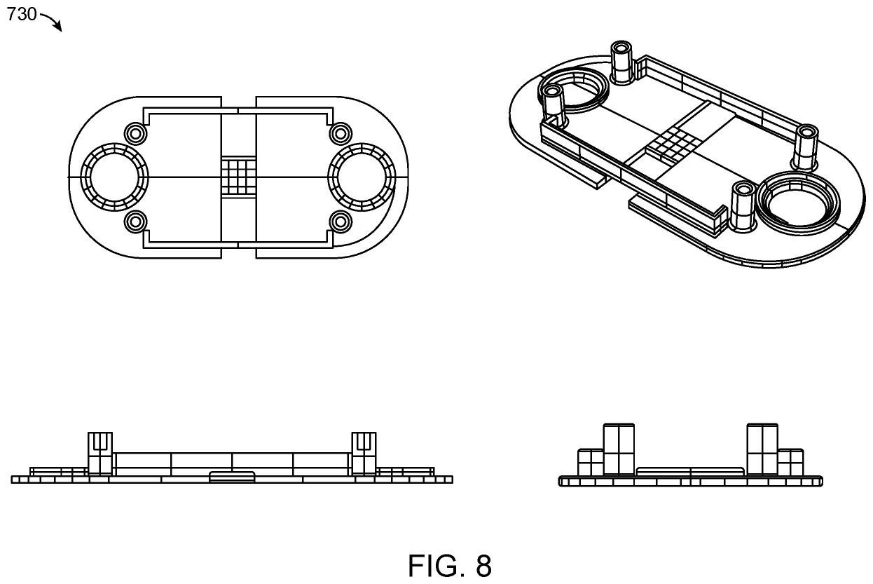

FIG. 8 illustrates an example of the shuttle 730 for the wearable device 110. The shuttle 730 integrates the electronic (battery 250 & printed circuit board (PCB) 710) and sensing components (flex sensor 220 & sEMG connectors 740) together. The two circular holes on either side may provide a snap connection for the sEMG connectors 740. The slots in the middle of the shuttle 730 fix the flex sensor 220 and prevent it from moving side to side. The raised walls create an encasing for the battery 250, while the posts allow the PCB 710 to be mounted above the battery using screws 720.

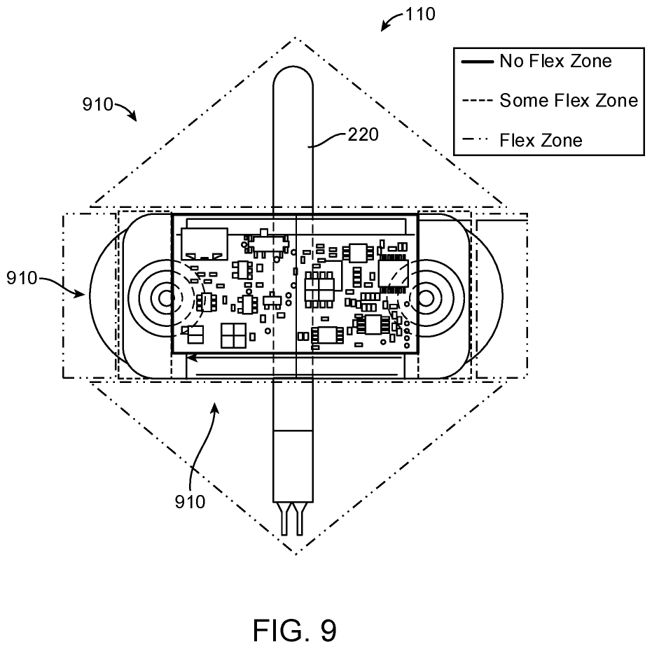

FIG. 9 illustrates the top view of the wearable device 110 with annotations for the flexible portions 910. In various examples, the device utilizes flex sensing and sits around the upper arm. Thus, the overall mechanical design of the wearable device 110 may incorporate curvature and flexibility. As illustrated in FIG. 9, the wearable device 110 may have zones of flexibility 910. The flex sensor may be positioned at the bottom of the shuttle and in between the two sEMG connectors. The flex sensor 220 may conform around the bicep while providing flexibility to work effectively. The accelerometer and gyroscope are integrated into one inertial motion unit IC that is placed on the PCB 710.

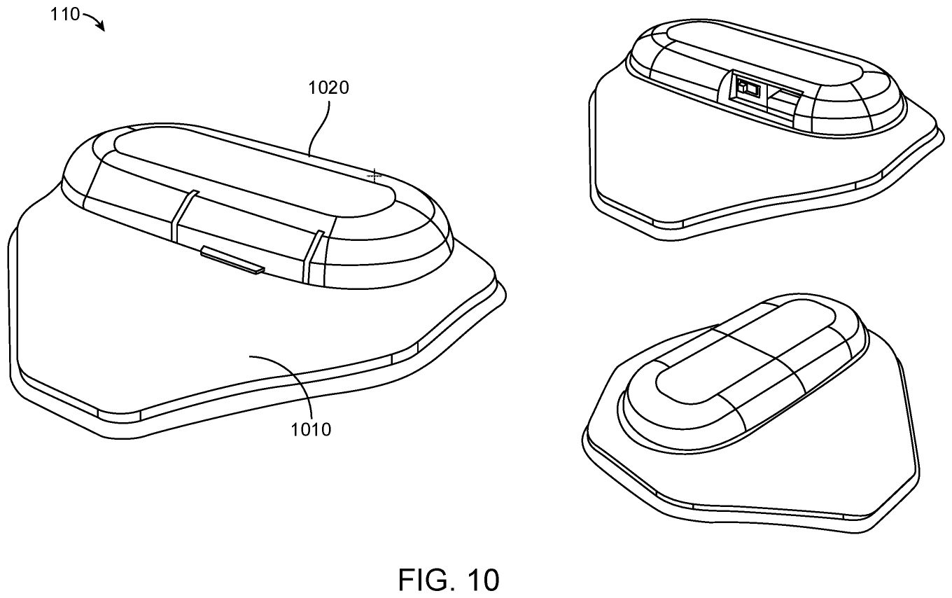

FIG. 10 illustrates a perspective view of the fully integrated wearable device 110. The components of the wearable device 110 may be composed of a flexible, comfortable silicone housing 1010. The left image shows the complete device, while the right-side images show the device without the silicone top housing 1020. On the top right image, one can see the cutout for the powering switch and micro-USB connector. A slight curvature of the main silicone housing 1010 will help the device fit nicely around the upper arm.

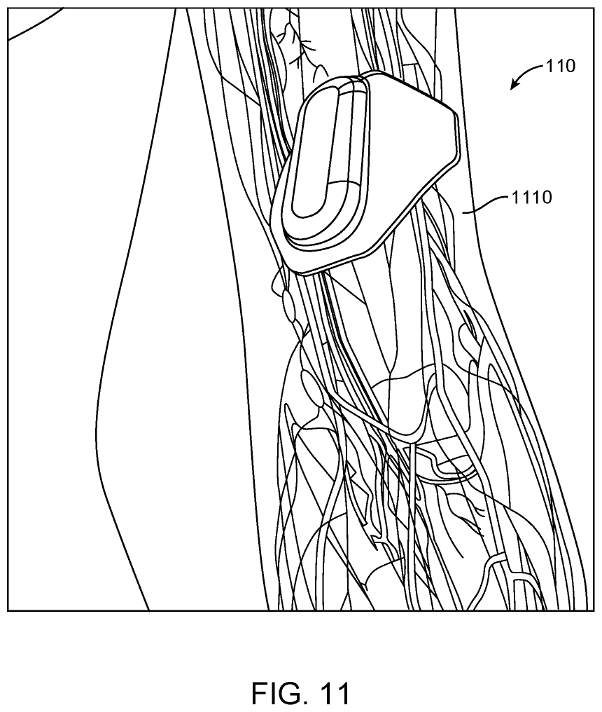

FIG. 11 illustrates an example of the wearable device 110 positioned on an arm 1110. By placing the device on the upper arm, the four signals relevant to motion and movement can be acquired. The sEMG electrodes 230 and flex sensors 220 measure local muscle activity of the bicep and arm, while the accelerometer and gyroscope measure motion and angular velocity both locally and globally. Locally is in reference to the exact site where the wearable device is placed on the arm 1110. The data acquired is a reflection of data both at this exact site but also of the entire body (globally). The flex sensor 220 is oriented perpendicular to the long head of the bicep, while the sEMG electrodes 230 are oriented parallel to it.

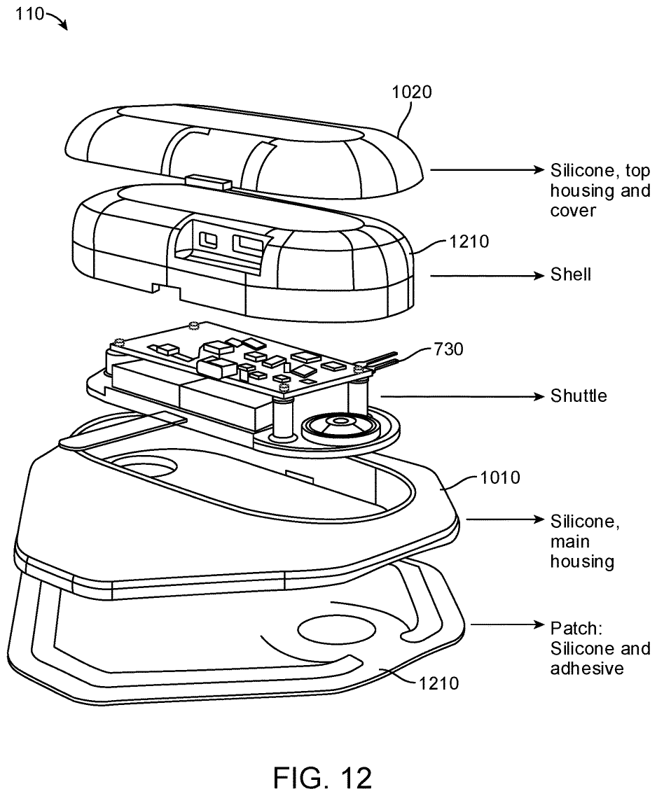

FIG. 12 illustrates an exploded view of an example of the wearable device 110. The shuttle 730 may integrate the electronic and sensing components (printed circuit board (PCB) 710, battery 250, flex sensor 220, and sEMG connectors 740). The shuttle 730 may include circular holes proximate to the ends of the shuttle 730 to create a snap connection for the sEMG connectors 740. Raised walls on the shuttle 730 may provide an encasing for the battery 250, while the posts allow the PCB 710 to be mounted above the battery 250 using screws 720. The shuttle 730 may further include slots centrally positioned to fix the flex sensor 220 and prevent it from moving side to side.

The flex sensor 220 may sit at the bottom of the shuttle 730 and in between the two sEMG connectors 740. The flex sensor 220 may conform around the bicep and provide sufficient flexibility to work effectively. The shuttle 730 may be made of PC ABS plastic or other suitable materials.

The wearable device 110 may further include a shell 1210 to encase the shuttle 730, a main silicone housing 1010 to hold the entire device, a top silicone housing 1020 to provide comfort to the user, and a replaceable silicone patch with double sided adhesive 1220 to attach the device to the user's arm. The shell 1210 may be also made of PC ABS plastic, or other suitable material(s). The shell 1210 may define an opening port to allow access to the power switch 257 and micro-USB B connector 258 for battery recharging.

The flex sensor 220 may sit inside the soft, flexible, silicone main housing 1010 that will allow it to be protected from the external environment and be mechanically secured within the wearable device 110, but with minimal weight and rigidity that could decrease its functionality. The shell 1210 and/or shuttle 730 may sit inside the silicone main housing 1010.

The logic illustrated in the flow diagrams may include additional, different, or fewer operations than illustrated. The operations illustrated may be performed in an order different than illustrated.

The system may be implemented with additional, different, or fewer components than illustrated. Each component may include additional, different, or fewer components.

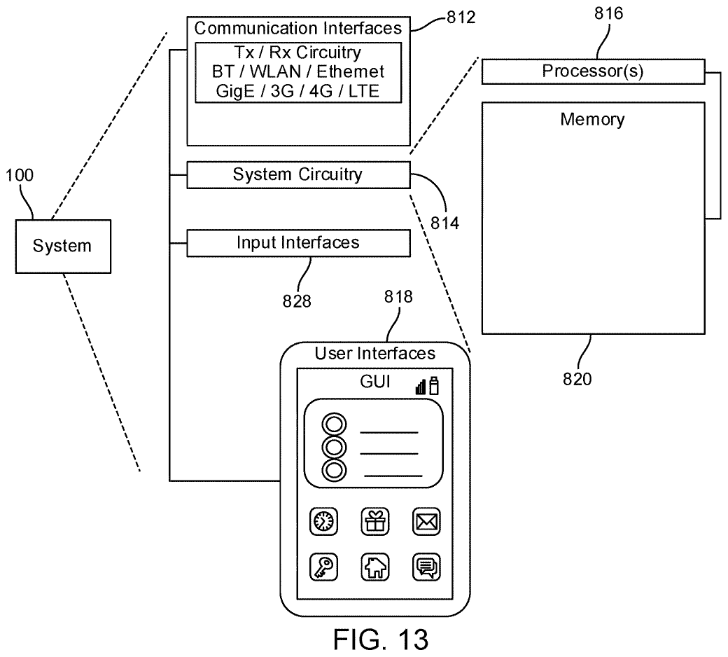

FIG. 13 illustrates a second example of the system 100. The system 100 may include communication interfaces 812, input interfaces 828 and/or system circuitry 814. The system circuitry 814 may include a processor 816 or multiple processors. Alternatively or in addition, the system circuitry 814 may include memory 820.

The processor 816 may be in communication with the memory 820. In some examples, the processor 816 may also be in communication with additional elements, such as the communication interfaces 812, the input interfaces 828, and/or the user interface 818. Examples of the processor 816 may include a general processor, a central processing unit, logical CPUs/arrays, a microcontroller, a server, an application specific integrated circuit (ASIC), a digital signal processor, a field programmable gate array (FPGA), and/or a digital circuit, analog circuit, or some combination thereof.

The processor 816 may be one or more devices operable to execute logic. The logic may include computer executable instructions or computer code stored in the memory 820 or in other memory that when executed by the processor 816, cause the processor 816 to perform the operations of the wearable device, the system device, the cloud infrastructure, the remote device, the seizure detection logic, and/or the system 100. The computer code may include instructions executable with the processor 816.

The memory 820 may be any device for storing and retrieving data or any combination thereof. The memory 820 may include non-volatile and/or volatile memory, such as a random access memory (RAM), a read-only memory (ROM), an erasable programmable read-only memory (EPROM), or flash memory. Alternatively or in addition, the memory 820 may include an optical, magnetic (hard-drive), solid-state drive or any other form of data storage device.

The user interface 818 may include any interface for displaying graphical information. The system circuitry 814 and/or the communications interface(s) 812 may communicate signals or commands to the user interface 818 that cause the user interface to display graphical information. Alternatively or in addition, the user interface 818 may be remote to the system 100 and the system circuitry 814 and/or communication interface(s) may communicate instructions, such as HTML, to the user interface to cause the user interface to display, compile, and/or render information content. In some examples, the content displayed by the user interface 818 may be interactive or responsive to user input. For example, the user interface 818 may communicate signals, messages, and/or information back to the communications interface 812 or system circuitry 814.

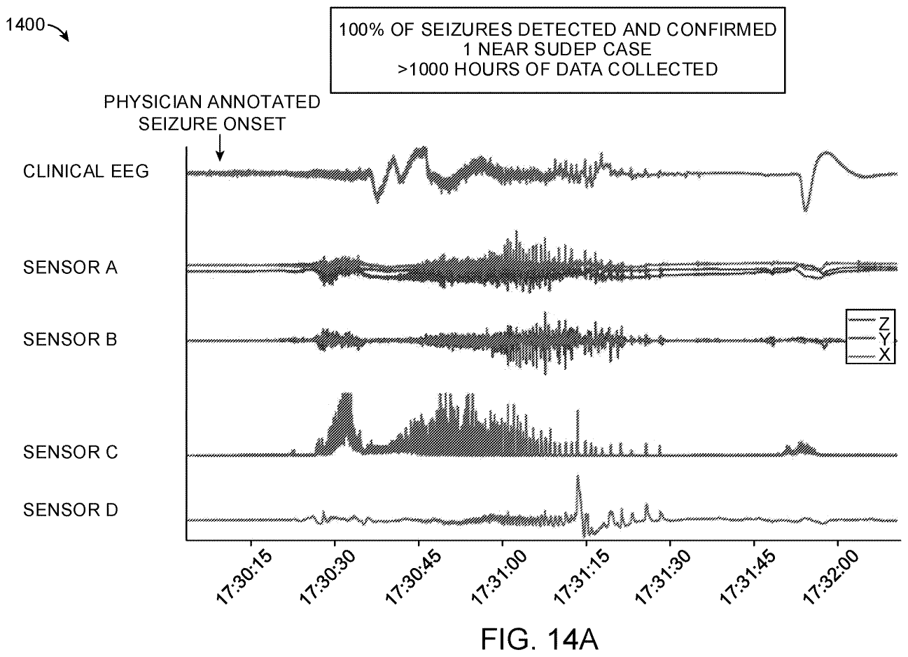

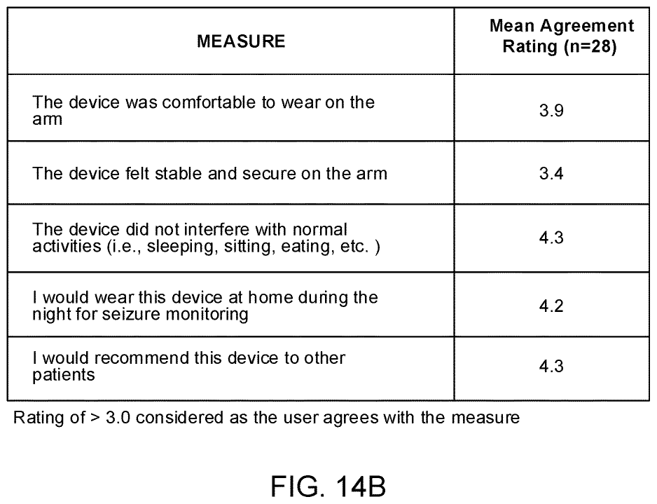

FIGS. 14A-14B illustrate results of a usability study conducted using examples of the wearable device 110 configured to provide a multimodal seizure sensor array. The usability study included over 1200 hours of operation of the wearable device 110 across 28 different patients 101. During the usability study, the wearable device 110 was able to detect and confirm all generalized tonic clonic seizure events that occurred among patients 101, including one confirmed near-SUDEP case.

FIG. 14A illustrates a graph 1400 of various sensor data measured by the wearable device 110 for a near SUDEP case that occurred during the study. As shown in the graph 1400, wearable device 110 sensor data (A-D) is presented for a patient 101 over time. This sensor data illustrates a near SUDEP case that occurred during the monitoring period, from the onset of symptoms to eventual stabilization. Also presented in graph 1400 is clinical EEG data that was measured from the patient 101 during the same time period. This EEG data was used as a control to verify the accuracy of the wearable device's 110 seizure detection and classification.

FIG. 14B illustrates mean agreement ratings for various measures that were proposed to the patients 101 in the usability study. These agreement ratings are a measurement of how much the patient 101 agreed with each of the measures, with ratings greater than “3” indicating that the patient agreed with the measure. Agreement ratings were averaged for each of the 28 patients 101 to determine a mean value. As illustrated in FIG. 14B, all measures had a generally positive agreement rating across the patients 101 of the study, with patients 101 agreeing that the wearable device 110 was “comfortable to wear,” “felt stable and secure,” and “did not interfere with normal activities.” Additionally, patients indicated that they would be inclined to use the wearable device 110 at night for seizure monitoring, and that they would recommend the wearable device to other patients 101.

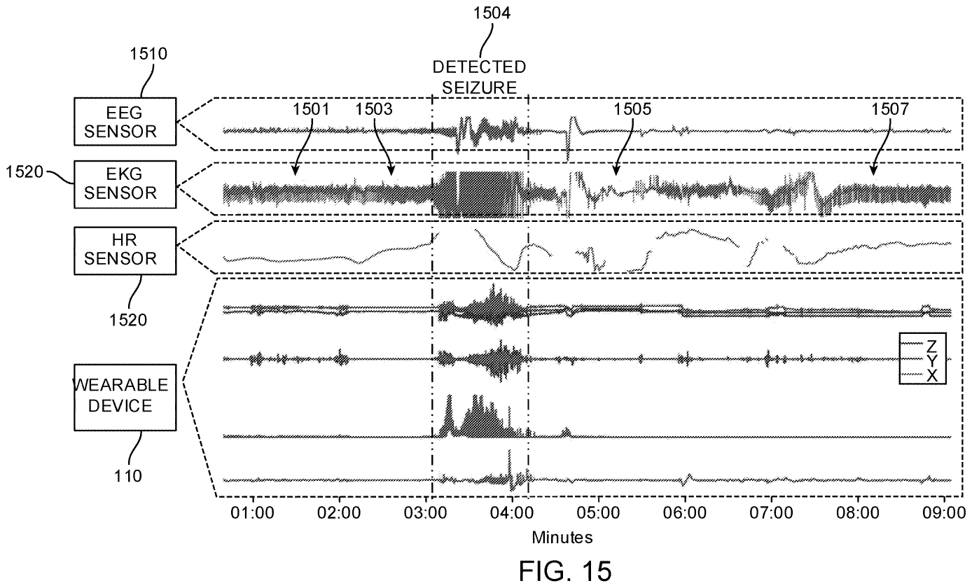

FIG. 15 illustrates results of a case study evaluating seizure detection capabilities of a wearable device 110 configured to provide a multimodal seizure sensor array. In this case, the wearable device 110 captured a near SUDEP seizure. This case illustrated the value of monitoring seizures and the induced cardiorespiratory dysfunctions. As illustrated in FIG. 15, the wearable device 110 captured sensor data from a patient 101 from a stable condition (1501) to an elevated heartrate (1503) leading to a convulsive seizure (1504) which resulted in cardiorespiratory collapse (1505). The patient 101 was then stabilized (1507). Because of the collapse, this patient's physicians are now recommending surgery instead of prescribing another anti-seizure medication. This represents a change in the patient's 101 clinical outcome driven by the use of the wearable device 110. If the patient 101 had experienced this seizure at home, and was using existing solutions, physicians would not have been informed of the cardiorespiratory collapse. With existing multimodal solutions, it is also still possible that seizure indicators would have also been missed. The multimodal techniques described herein that utilize cardiac and respiratory signals are more effective at detecting and appropriately classifying seizures in outpatient environments.

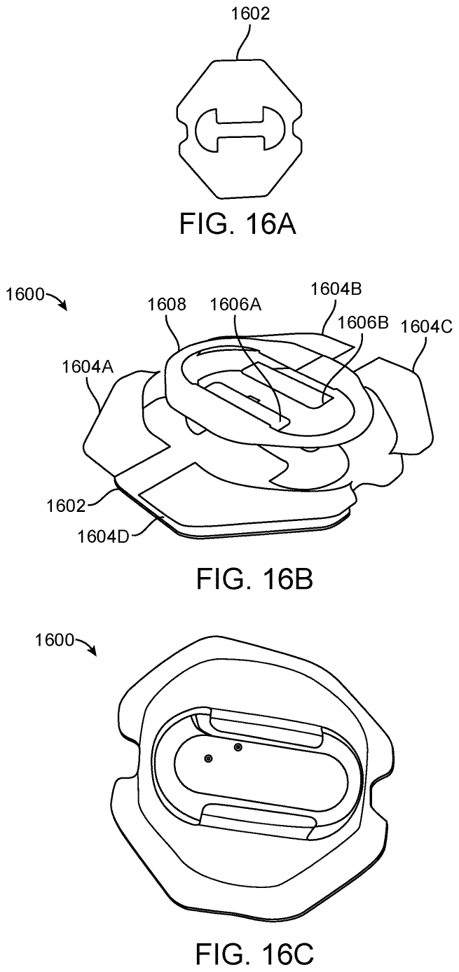

FIGS. 16A-C illustrate configurations of an alternative adhesive pad 1600 for placing a wearable device 110 onto a body of a patient 101. In contrast to the adhesive pad 1220 illustrated in FIG. 12, alternative adhesive pad 1600 is designed to be applied on top of the wearable device top housing 1020 and main silicone housing 1010 in order to allow the wearable device to conform to smaller arm sizes (e.g., in pediatric cases). In this implementation, adhesive pad 1600 includes various hardware to facilitate the attachment of the adhesive pad 1600 to the wearable device top housing 1020 and main silicone housing 1010, for example, patch 1602, backer sections 1604A-D, and an optional stiffener ring 1608 with adhesives 1606A-B. In some implementations, backer sections 1604-D and stiffener ring 1608 are laser-cut backers to improve usability of the patch (e.g., to increase ease-of-user by a user). In some implementations, the wearable device 110 may be attached to a body using at least one of patches 1220 or adhesive pad 1600 (i.e., use of wearable device 110 does not independently require patches 1220 and adhesive pad 1600 for attachment).

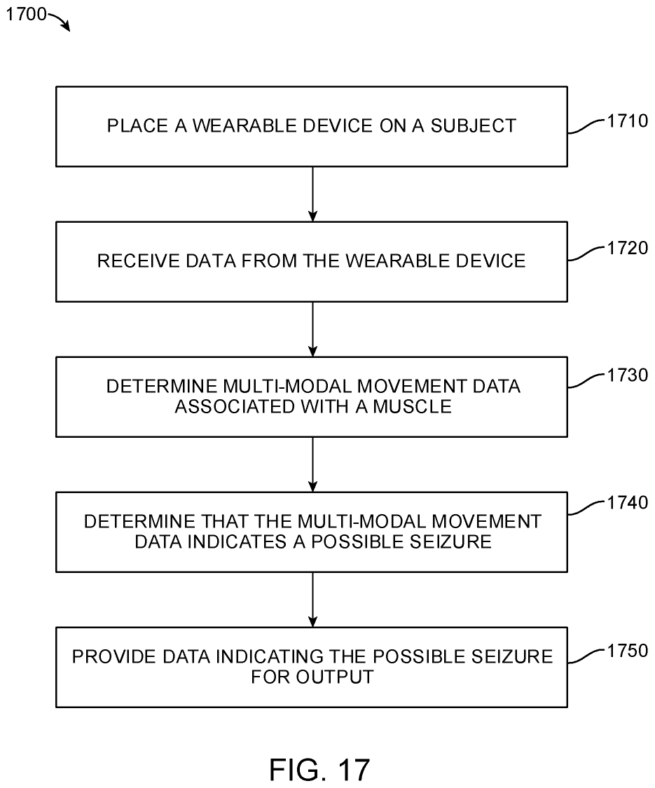

FIG. 17 illustrates an example of a process 1700 for detecting a possible seizure using a wearable device configured to provide a multimodal seizure sensor array. The process 1700 includes placing the wearable device 110 on a subject 101 (1710). Placing the wearable device 110 on a subject can include using the various adhesive patches described above, for example, as described in FIGS. 12 and 16A-C. The process 1700 includes receiving data from the wearable device 110 (1720). This data can be determined using the methods described above, for example, as described in FIGS. 2-4. This data can be transmitted by the wearable device 110 to other devices in the system 100, for example, patient device 120 or other remote devices 130. The process 1700 includes determining multimodal movement data associated with a muscle (1730). This determination can be conducted as described above using an appropriate combination of sensors, for example, the flex sensor and sEMG as described in FIG. 6. The process 1700 includes determining that the multimodal movement data indicates a possible seizure (1740). This determination can be made, for example, by extracting relevant features from the multimodal movement data and classifying these features as to whether they indicate a seizure condition as described in FIG. 6. The process 1700 includes providing data indicating the possible seizure for output (1750). This output can be provided to the patient device 120 or a remote device 130. Alternatively, or in addition, the wearable device 110 can also provide output this output directly.

The system 100 may be implemented in many different ways. In some examples, the system 100 may be implemented with one or more logical components. For example, the logical components of the system 100 may be hardware or a combination of hardware and software. The logical components may include the wearable device, the system device, the cloud infrastructure, the remote device, the seizure detection logic, or any component or subcomponent of the system 100. In some examples, each logic component may include an application specific integrated circuit (ASIC), a Field Programmable Gate Array (FPGA), a digital logic circuit, an analog circuit, a combination of discrete circuits, gates, or any other type of hardware or combination thereof. Alternatively or in addition, each component may include memory hardware, such as a portion of the memory 820, for example, that comprises instructions executable with the processor 816 or other processor to implement one or more of the features of the logical components. When any one of the logical components includes the portion of the memory that comprises instructions executable with the processor 816, the component may or may not include the processor 816. In some examples, each logical component may just be the portion of the memory 820 or other physical memory that comprises instructions executable with the processor 816, or other processor(s), to implement the features of the corresponding component without the component including any other hardware. Because each component includes at least some hardware even when the included hardware comprises software, each component may be interchangeably referred to as a hardware component.

Some features are shown stored in a computer readable storage medium (for example, as logic implemented as computer executable instructions or as data structures in memory). All or part of the system and its logic and data structures may be stored on, distributed across, or read from one or more types of computer readable storage media. Examples of the computer readable storage medium may include a hard disk, a flash drive, a cache, volatile memory, non-volatile memory, RAM, flash memory, or any other type of computer readable storage medium or storage media. The computer readable storage medium may include any type of non-transitory computer readable medium, a volatile memory, a non-volatile memory, ROM, RAM, or any other suitable storage device.

The processing capability of the system may be distributed among multiple entities, such as among multiple processors and memories, optionally including multiple distributed processing systems. Parameters, databases, and other data structures may be separately stored and managed, may be incorporated into a single memory or database, may be logically and physically organized in many different ways, and may be implemented with different types of data structures such as linked lists, hash tables, or implicit storage mechanisms. Logic, such as programs or circuitry, may be combined or split among multiple programs, distributed across several memories and processors, and may be implemented in a library, such as a shared library (for example, a dynamic link library (DLL).

All of the discussion, regardless of the particular implementation described, is illustrative in nature, rather than limiting. For example, although selected aspects, features, or components of the implementations are depicted as being stored in memory(s), all or part of the system or systems may be stored on, distributed across, or read from other computer readable storage media, for example, secondary storage devices such as hard disks, flash memory drives, floppy disks, and CD-ROMs. Moreover, the various logical units, circuitry and screen display functionality is but one example of such functionality and any other configurations encompassing similar functionality are possible.

The respective logic, software or instructions for implementing the processes, methods and/or techniques discussed above may be provided on computer readable storage media. The functions, acts or tasks illustrated in the figures or described herein may be executed in response to one or more sets of logic or instructions stored in or on computer readable media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one example, the instructions are stored on a removable media device for reading by local or remote systems. In other examples, the logic or instructions are stored in a remote location for transfer through a computer network or over telephone lines. In yet other examples, the logic or instructions are stored within a given computer and/or central processing unit (“CPU”).

Furthermore, although specific components are described above, methods, systems, and articles of manufacture described herein may include additional, fewer, or different components. For example, a processor may be implemented as a microprocessor, microcontroller, application specific integrated circuit (ASIC), discrete logic, or a combination of other types of circuits or logic. Similarly, memories may be DRAM, SRAM, Flash or any other type of memory. Flags, data, databases, tables, entities, and other data structures may be separately stored and managed, may be incorporated into a single memory or database, may be distributed, or may be logically and physically organized in many different ways. The components may operate independently or be part of a same apparatus executing a same program or different programs. The components may be resident on separate hardware, such as separate removable circuit boards, or share common hardware, such as a same memory and processor for implementing instructions from the memory. Programs may be parts of a single program, separate programs, or distributed across several memories and processors.

A second action may be said to be “in response to” a first action independent of whether the second action results directly or indirectly from the first action. The second action may occur at a substantially later time than the first action and still be in response to the first action. Similarly, the second action may be said to be in response to the first action even if intervening actions take place between the first action and the second action, and even if one or more of the intervening actions directly cause the second action to be performed. For example, a second action may be in response to a first action if the first action sets a flag and a third action later initiates the second action whenever the flag is set.

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed.

While various embodiments have been described, it will be apparent to those of ordinary skill in the art that many more embodiments and implementations are possible. Accordingly, the embodiments described herein are examples, not the only possible embodiments and implementations.

The drawings include various texts which are incorporated by reference herein to supplement this written description. Thus, for the purposes of this provisional application, all text included in the drawings is intended to increase understanding of the system and methods described herein.

What is claimed is:

1. A system comprising:
a wearable device comprising:
a flex sensor;
a plurality of surface electromyogram (sEMG) electrodes; and
circuitry configured to:
measure, sEMG activity of a muscle proximate to the sEMG electrodes based on sEMG signals received from the sEMG electrodes;
measure movement of the muscle based on signals received from the flex sensor;
measure acceleration and angular velocity based on signals generated by an inertial motion unit (IMU);
wirelessly communicate the measured sEMG activity, the measured muscle movement, and the measured acceleration and angular velocity.

2. The system of claim 1, further comprising
a remote device configured to:
receive the inertial motion measurements, the measured sEMG activity, and the measured acceleration and angular velocity;
determine, based on the received measurements, a seizure is occurring in a person wearing the wearable device; and
output a message in response to determination of the seizure.

3. The system of claim 1, wherein to determine, based on the received signals, a seizure is occurring in a person wearing the wearable device, the processor of the remote device is further configured to:
identify features based on the inertial motion measurements, the measured sEMG activity, and the measured muscle movement; and
classify the features as a seizure event.

4. The system of claim 1, wherein the IMU comprises a 3-axis gyroscope and a 3-axis accelerometer.

5. The system of claim 1, wherein to measure, based on the sEMG signals received from the sEMG electrodes, sEMG activity of a muscle proximate to the sEMG electrodes, the circuitry is further configured to:
receive the signals from the sEMG electrodes;
filter the signals, wherein the filtered signals from the sEMG electrodes have a frequency between 100 Hz and 300 Hz.

6. The system of claim 5, wherein the circuitry is further configured to:
determine the root-mean-square (RMS) of the filtered signals.

7. The system of claim 1, wherein to measure, based on signals received from the flex sensor, movement of a muscle proximate to the flex sensors, the circuitry is further configured to:
generate an electrical signals based on the voltage of the flex sensor;
filter the signals, wherein the filtered signals from the flex sensor signals have a frequency between 0.1-50 Hz

8. The system of claim 1, wherein to measure acceleration and angular velocity based on signals generated by an IMU, the circuitry is further configured to:
generate a first acceleration vector parallel to the plane of the bed and a second acceleration vector perpendicular to the plane of the bed.

9. The system of claim 1, wherein the flex sensor is positioned in between the two electrodes, wherein the flex sensor and electrodes are flexible and conform to a surface of an arm along a muscle.

10. The system of claim 9, wherein the flex sensor is elongated at curves about the bicep along a direction substantially perpendicular to a long head of the muscle and the sEMG electrodes are oriented in a direction parallel to the long head of the muscle.

11. The system of claim 1, wherein the wearable device further comprises a shuttle which receives the sEMG electrodes, flex sensor, and circuitry.

12. The system of claim 11, wherein the wearable device further comprises a housing configured to conform and flex to a muscle, wherein the shuttle is disposed in the housing and at least a portion of the sEMG electrodes outside of the housing for contact with the arm.

13. The system of claim 12, wherein the flex sensor is disposed inside of the housing, wherein flexure of the housing causes flexure of the flex sensor.

14. The system of claim 12, further comprising an adhesive layer attached to an outer surface of the housing, wherein the adhesive layer is configured to attach the wearable device to an arm.

15. The system of claim 12, wherein the muscle is a bicep of an arm.

16. A wearable device comprising:
a flex sensor configured to collect one or more first signals;
an inertial motion unit (IMU) configured to collect one or more second signals;
a plurality of surface electromyogram (sEMG) electrodes configured to (i) be placed approximate to a muscle of a subject and (ii) collect one or more third signals; and
a microprocessor configured to perform operations comprising:
determine multimodal movement data of the muscle based on the one or more first signals, the one or more second signals, and the one or more third signals, wherein the multimodal movement data specifies at least an acceleration associated with movement of the muscle, an angular velocity associated with movement of the muscle, and sEMG activity associated with the movement of the muscle,
determine whether the multimodal movement data indicates a possible seizure, and
provide, for output, data indicating whether the movement indicates a possible seizure.

17. The apparatus of claim 16, wherein the muscle comprises an arm muscle.

18. The apparatus of claim 16, wherein the muscle comprises a leg muscle.

19. The apparatus of claim 16, wherein the muscle comprises a torso muscle.

20. The apparatus of claim 16, wherein determining whether the multimodal movement data indicates a possible seizure comprises:
processing the first set of signals, the second set of signals, and the third set of signals;
identifying a set of features associated with the multimodal movement data based on processing the first set of signals, the second set of signals, and the third set of signals;
determining that the set of features include a predetermined feature associated with a seizure condition; and
determining that the multimodal movement data indicates the possible seizure based on determining that the set of features include the predetermined feature.

21. A method comprising:
placing the wearable device on a subject, wherein the wearable device comprises (i) a flex sensor, (ii) an inertial motion unit (IMU) sensor, and (iii), a plurality of surface electromyogram (sEMG) electrodes;
receiving data indicating (i) one or more first signals from the flex sensor, (ii) one or more second signals from the IMU sensor, and (iii) one or more third signals from the plurality of sEMG electrodes;
determining multimodal movement data of the muscle based on the one or more first signals, the one or more second signals, and the one or more third signals, wherein the multimodal movement data specifies at least an acceleration associated with movement of the muscle, an angular velocity associated with movement of the muscle, and sEMG activity associated with the movement of the muscle;
determining that the multimodal movement data indicates a possible seizure;
providing, for output, data indicating the possible seizure.

22. The method of claim 21, placing the wearable device on the subject comprises placing the wearable device on an arm of the subject.

23. The method of claim 21, placing the wearable device on the subject comprises placing the wearable device on a leg of the subject.

24. The method of claim 21, placing the wearable device on the subject comprises placing the wearable device on a torso of the subject.
